(19) United States
(12) Patent Application Publication
(10) Pub. No.: US 2008/0143826 A1
(43) Pub. Date: Jun. 19, 2008

(54) IMAGE-SIGNAL TRANSMISSION SYSTEM, ELECTRONIC ENDOSCOPE, AND ENDOSCOPE PROCESSOR

(75) Inventor: Yuichi Shibasaki, Saitama (JP)

Correspondence Address: GREENBLUM & BERNSTEIN, P.L.C., 1950 ROLAND CLARKE PLACE, RESTON, VA, 20191, US

(73) Assignee: PENTAX CORPORATION, Tokyo (JP)

(21) Appl. No.: 11/956,576

(22) Filed: Dec. 14, 2007

(30) Foreign Application Priority Data
Dec. 15, 2006 (JP) 2006-338281

(51) Int. Cl. H04N 7/18

(52) U.S. Cl. 348/65; 348/E07.085

(57) ABSTRACT

An image-signal transmission system comprising an imaging device, a transmitter, and a determination block, is provided. The imaging device outputs first pixel signals at a first frequency. The first pixel signals vary according to the amount of light received by pixels. A plurality of the pixels are arranged in two dimensions on a light-receiving surface of the imaging device. The transmitter transmits the first pixel signal copies separately, several times at a second frequency. The second frequency is two or more times higher than the first frequency. The determination block receives the first pixel signal copies from the transmitter. The determination block determines whether noise is mixed into the received first pixel signals by comparing the first pixel signal copies.

### BACKGROUND OF THE INVENTION

1. Field of the Invention

The present invention relates to an image-signal transmission system that reduces the influence of external noise mixed into an image signal under transmission.

2. Description of the Related Art

An electronic endoscope may be used to observe the internal structure of an organism, a mechanism, or other structure. An electronic endoscope has an imaging device at the head end of an insertion tube. An image signal generated by the imaging device is sent to an endoscope processor via a transmission cable.

External noise may contaminate the image signal at different points in the transmission path from the head end of the insertion tube to the endoscope processor. Degraded image quality remains problematic due to contamination by external noise.

In order to prevent external noise from mixing into a signal, some inventions have been proposed. For example, Japanese Patent Publication No. 2003-61901 discloses a transmission cable inserted into a flexible ferromagnetic tube. Japanese Patent Publication No. 2002-177215 discloses a transmission cable covered with a conductive shield sheet.

However, even if the above inventions are applied, it is difficult to reliably prevent external noise from mixing into the transmitted signal. It is still desired to protect an image quality from degradation due to external noise.

### SUMMARY OF THE INVENTION

Therefore, an object of the present invention is to provide an image-signal transmission system that lowers the influence of external noise mixed into a transmitted signal along its transmission path.

According to the present invention, an image-signal transmission system comprising an imaging device, a transmitter, and a determination block, is provided. The imaging device output first pixel signals at a first frequency. The first pixel signals vary according to the amount of light received by pixels. A plurality of the pixels are arranged in two dimensions on a light-receiving surface of the imaging device. The transmitter transmits the first pixel signal copies separately several times at a second frequency. The second frequency is two or more times higher than the first frequency. The determination block receives the first pixel signal copies from the transmitter. The determination block determines whether noise is mixed into the received first pixel signals by comparing the first pixel signal copies.

Further, the determination block determines that the noise is mixed into the received first pixel signal if signal levels of a plurality of the first pixel signal copies are different.

Further, the image-signal transmission system comprises an interpolation block. The interpolation block generates a second pixel signal using the received first pixel signals corresponding to the pixels surrounding a focused pixel. The focused pixel is a pixel which the determination block determines is noisy. The interpolation clock replaces the received first pixel signal by the second pixel signal.

Or, the image-signal transmission system comprises an interpolation block that generates a third pixel signal using a fourth pixel signal from the same focused pixel. The focused pixel is a pixel whose first pixel signal was determined to be noisy by the determination block. The fourth pixel signal is generated before and/or after the first pixel signal. The interpolation block replaces the first pixel signal by the third pixel signal.

Or, the determination block outputs a fifth pixel signal among the first pixel signal copies of the same pixel where the determination block determines the noise is mixed into the received first pixel signal. The fifth pixel signal is one among a plurality of the first pixel signal copies. The number of the fifth pixel signals whose signal levels are substantially equal to each other is the greatest in the first pixel signal copies.

### BRIEF DESCRIPTION OF THE DRAWINGS

The objects and advantages of the present invention will be better understood from the following description, with reference to the accompanying drawings in which:

FIG. 1 is an outside view of an endoscope system having an image-signal transmission system as a first embodiment of the present invention;

FIG. 2 is a block diagram showing the internal structure of the electronic endoscope of the first embodiment;

FIG. 3 is a timing chart showing the pixel signal output from the imaging device, the pixel signal being written to the first and second storage areas, operations of the first and second storage areas, and the pixel signal being read and transmitted by the transmission-rate modification circuit at successive moments in time in the first embodiment;

FIG. 4 is a block diagram showing the internal structure of the endoscope processor of the first embodiment;

FIG. 5 is a timing chart showing operations of the first RAM and pixel signals read by the memory controller at successive moments in time in the first embodiment;

FIG. 6 shows the arrangement of a focused pixel and the pixels which surround the focused pixel and are used for interpolation processing;

FIG. 7 is a flowchart describing the process for controlling each component of the endoscope system with the system controller and the timing generator for reducing the influence of noise in the first embodiment;

FIG. 8 is a block diagram showing the internal structure of the electronic endoscope of the second embodiment;

FIG. 9 is a block diagram showing the internal structure of the endoscope processor of the second embodiment;

FIG. 10 is a flowchart describing the process for controlling each component of the endoscope system with the system controller and the timing generator for reducing the influence of noise in the second embodiment;

FIG. 11 is a timing chart showing the pixel signal output from the imaging device and the pixel signal read and output by the transmission-rate modification circuit at successive moments in time in other embodiments based on the first and second embodiments; and

FIG. 12 is a conceptual diagram used to explain interpolation processing using the time filter.

### DESCRIPTION OF THE PREFERRED EMBODIMENTS

The present invention is described below with reference to the first and second embodiments shown in the drawings.

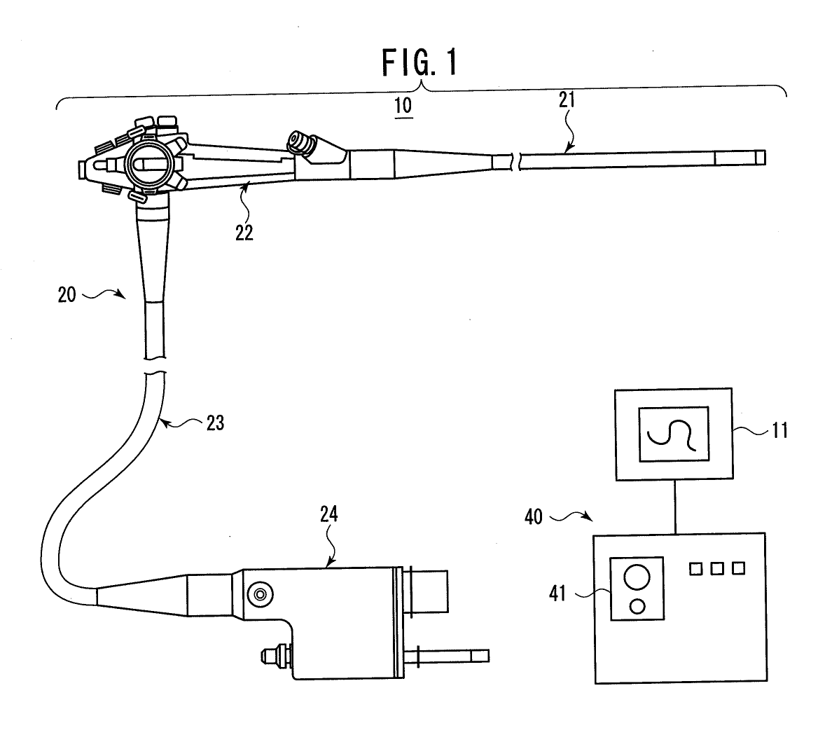

In FIG. 1, an endoscope system 10 of the first embodiment comprises an electronic endoscope 20, an endoscope processor 40, and a monitor 11. The endoscope processor 40 is connected to the electronic endoscope 20 and the monitor 11.

A part of the electronic endoscope 20 is inserted into a body. The electronic endoscope captures an optical image in the body. An image signal corresponding to the captured optical image is generated. The image signal is transmitted to the endoscope processor 40. The endoscope processor 40 carries out predetermined signal processing on the received image signal. The image signal having undergone predetermined signal processing is transmitted to the monitor 11, where an image corresponding to the image signal is displayed.

The electronic endoscope comprises an insertion tube 21, a control block 22, a connection tube 23, and a connector 24. The base end of the insertion tube is connected to the control block 22. The control block 22 is connected to the connector 24 via the connection tube 23.

The insertion tube is flexible, and may be inserted into an internal body or an internal mechanism. The control block 22 has switches for initiating some functions of the electronic endoscope 20 and the endoscope system 10. The electronic endoscope 20 is connected to the endoscope processor 40 by inserting the connector 24 into the terminal 41 of the endoscope processor 40.

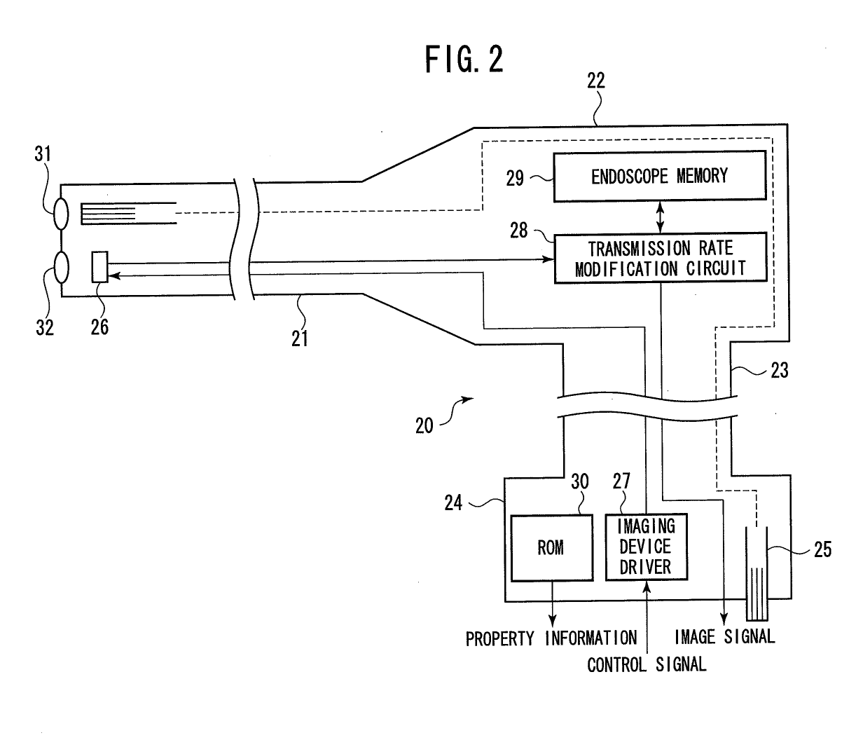

The internal structure of the electronic endoscope 20 is explained with reference to FIG. 2. The electronic endoscope 20 comprises a light guide 25, an imaging device 26, an imaging device driver 27, a transmission-rate modification circuit (transmitter) 28, an endoscope memory 29, a ROM 30, and other components.

The light guide 25 is a bundle of optical fibers, of which one end is mounted in the connector 24 and the other end is mounted at the head end of the insertion tube 21. The imaging device 26 is mounted at the head end of the insertion tube 21. The imaging device driver 27 and the ROM are mounted in the connector 24. The transmission-rate modification circuit 28 and the endoscope memory 29 are mounted in the control block 22.

When the connector 24 is inserted into the terminal 41, the light guide 25 is optically connected to the light-source unit (not depicted in FIG. 2) mounted in the endoscope processor 40. Illumination light emitted by the light-source unit is transmitted by the light guide 25. The illumination light transmitted to the exit end of the light guide 25 illuminates a peripheral area around the head end of the insertion tube 21 after passing through a diffuser lens 31.

An optical image of the illuminated subject is incident onto the light-receiving surface of the imaging device 26 after passing through an object lens 32. The imaging device 26 is a CCD imaging device or a CMOS imaging device. The imaging device driver 27 drives the imaging device 26 so that the imaging device 26 generates an image signal corresponding to the optical image incident on the imaging device 26. The image signal is generated every 1/30 second. The time necessary for the imaging device driver 27 to drive the imaging device 26 is controlled by a timing generator (not depicted in FIG. 2) mounted in the endoscope processor 40.

A plurality of pixels (not depicted) are arranged in two dimensions on the light-receiving surface of the imaging device 26. Each pixel generates a pixel signal according to the amount of light it receives. Pixel signals from each pixel arranged on the light-receiving surface are output separately in order. In addition, the pixel signals are output at a 13.5 MHz transmission rate (the first frequency). The image signal comprises a plurality of pixel signals generated by a plurality of pixels arranged on the light-receiving surface.

The pixel signals output from the imaging device 26 are input to the transmission-rate modification circuit 28 in order. The pixel signals input to the transmission-rate modification circuit 28 are stored in the endoscope memory 30. The pixel signals stored in the endoscope memory 30 are read by the transmission-rate modification circuit 28. The transmission-rate modification circuit 28 outputs the pixel signals to the endoscope processor 40 with a modified transmission rate of 27 MHz (the second frequency).

The transmission-rate modification circuit 28 output the same pixel signals twice, as explained below.

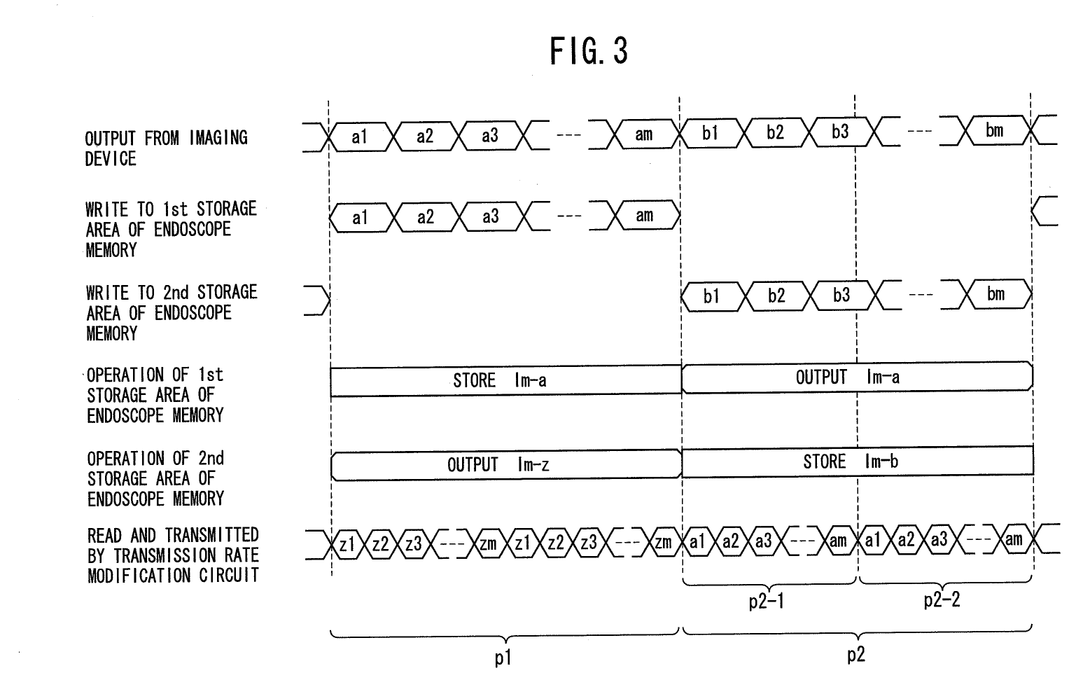

During the period p1, first through mth (m, a positive integer) pixel signals (al through am in the row “Output from imaging device” in FIG. 3) are output from the first through mth pixels. The ath frame of an image signal, hereinafter referred to as Im-a, is comprised of a set of pixel signals that are output from the imaging device 26 during the period p1. During the period p2 after period p1, first through mth pixel signals (b1 through bm in the row “Output from imaging device” in FIG. 3) are output from the first through mth pixels. The bth frame of an image signal, hereinafter referred to as Im-b, is comprised of a set of pixel signals that are output from the imaging device 26 during period p2.

The pixel signals transmitted from the imaging device 26 to the transmission-rate modification circuit 28 is received and written to the endoscope memory 29 substantially at the same time as the pixel signals are output from the imaging device 26. The endoscope memory 29 has a first and second storage areas (not depicted). Each of the first and second storage areas can store one frame of an image signal.

While a frame of an image signal gets written in one of the storage areas, an image signal already stored in the other storage area is read by the transmission-rate modification circuit 28. In the next cycle, the next frame of the image signal gets written to the other storage area, and the previous frame of the image signal is read by the transmission-rate modification circuit 28. From then on, pixel signals are repeatedly written to, and read from one of the storage areas. At the same time, pixel signals are repeatedly read from, and written to the other storage area.

For example, during period p1, the pixel signals that Im-a comprises are stored in the first storage area (a1 through am in the row “Write to first storage area” in FIG. 3). During period p2, the pixel signals that Im-b comprises are stored in the second storage area (b1 through bm in the row “Write to second storage area” in FIG. 3).

Im-a is read by the transmission-rate modification circuit 28 during period p2 (row “Operation of first storage area” in FIG. 3). Im-b is read by the transmission-rate modification circuit 28 during period p3 (not depicted in FIG. 3) after period p2.

The transmission-rate modification circuit 28 reads pixel signals from the first or second storage areas, in order, at a 27 MHz transmission rate. The read pixel signals are transmitted to the endoscope processor 40 at the same transmission rate as they are read from the first and second storage areas. After outputting the pixel signals for one frame of an image signal, the same pixel signals stored in the same storage area are read again and transmitted to the endoscope processor 40. Thus, the same pixel signals are transmitted twice.

For example, during both first and second halves of period p2, the pixel signals that Im-a comprises are read by the transmission-rate modification circuit 28 and transmitted to the endoscope processor 40 (see p2-1, p2-2 in the row “Read and transmitted” in FIG. 3). Similarly, during both first and second halves of period p3, the pixel signals that Im-b comprises are read by the transmission-rate modification circuit 28 and transmitted to the endoscope processor 40.

The ROM 30 stores property information that is specific to each electronic endoscope 20. As described below, the property information is transmitted to the endoscope processor 40. The endoscope processor 40 uses the property information for carrying out predetermined functions and predetermined signal processing.

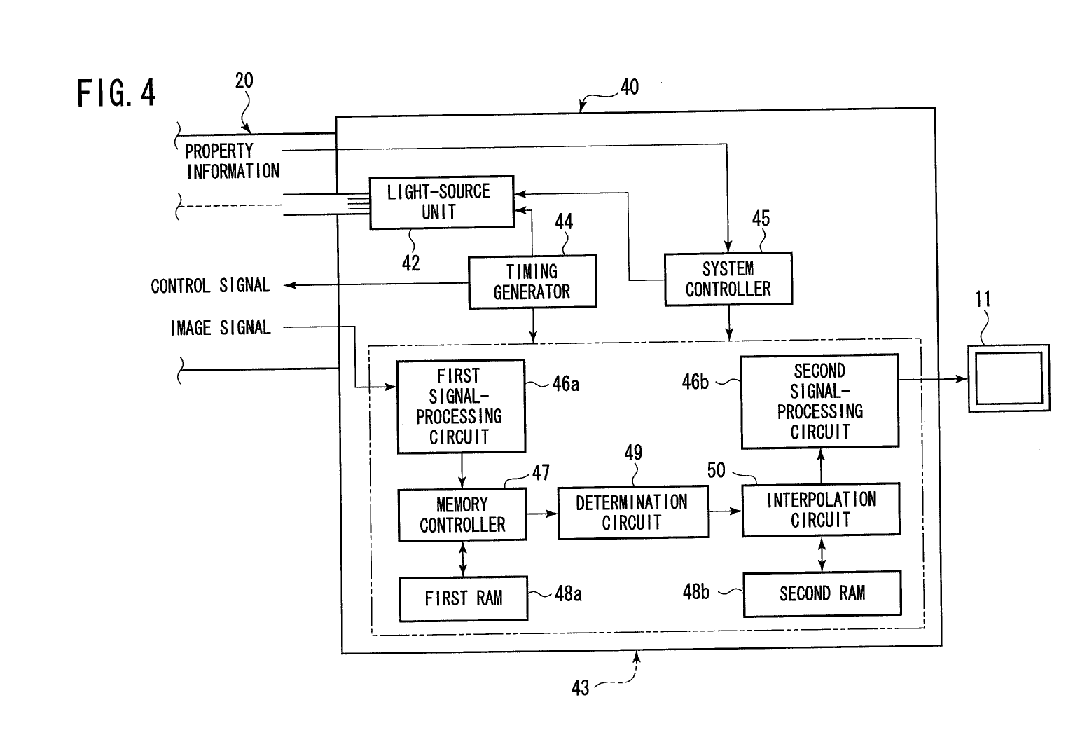

Next, the internal structure of the endoscope processor is explained with reference to FIG. 4. The endoscope processor 40 comprises the light-source unit 42, a signal-processing unit 43, the timing generator 44, a system controller 45, and other components.

As described above, the light-source unit 42 is optically connected to the light guide 25, and supplies the illumination light for illuminating a subject. The light-source unit 42 has a diaphragm (not depicted) and a shutter (not depicted). The aperture ratio of the diaphragm and the operation of opening and closing the shutter is controlled by the timing generator 44 and the system controller 45.

When the connector 24 of the endoscope processor 20 is connected to the endoscope processor 40, the transmission-rate modification circuit 28 and the signal-processing unit 43 communicate electrically with each other. The image signal output from the transmission-rate modification circuit 28 is transmitted to the signal-processing unit 43.

The signal-processing unit 43 comprises first and second signal-processing circuits 46a and 46b, a memory controller 47, first and second RAMs 48a and 48b, a determination circuit 49, and an interpolation circuit 50. The first signal-processing circuit 46a, the memory controller 47, the determination circuit 49, the interpolation circuit 50, and the second signal-processing circuit 46b communicate serially. The first and second RAMs 48a and 48b are connected to the memory controller 47 and the interpolation circuit 50, respectively.

The pixel signals transmitted from the electronic endoscope 20 to the signal-processing unit 43 are received by the first signal-processing circuit 46a. The first signal-processing circuit carries out predetermined signal processing, such as A/D conversion, color interpolation, and so on, on the received pixel signals.

The pixel signals, having undergone predetermined signal processing, are output to the memory controller 47. As described above, pixel signals constituting a given frame of an image signal are transmitted twice from the electronic endoscope 20. The transmitted pixel signals are stored in the first RAM 48a.

The first RAM 48a has first and second storage areas. Each of the storage areas can store two frames of an image signal. The pixel signals that are transmitted twice are stored in different parts of the same storage area.

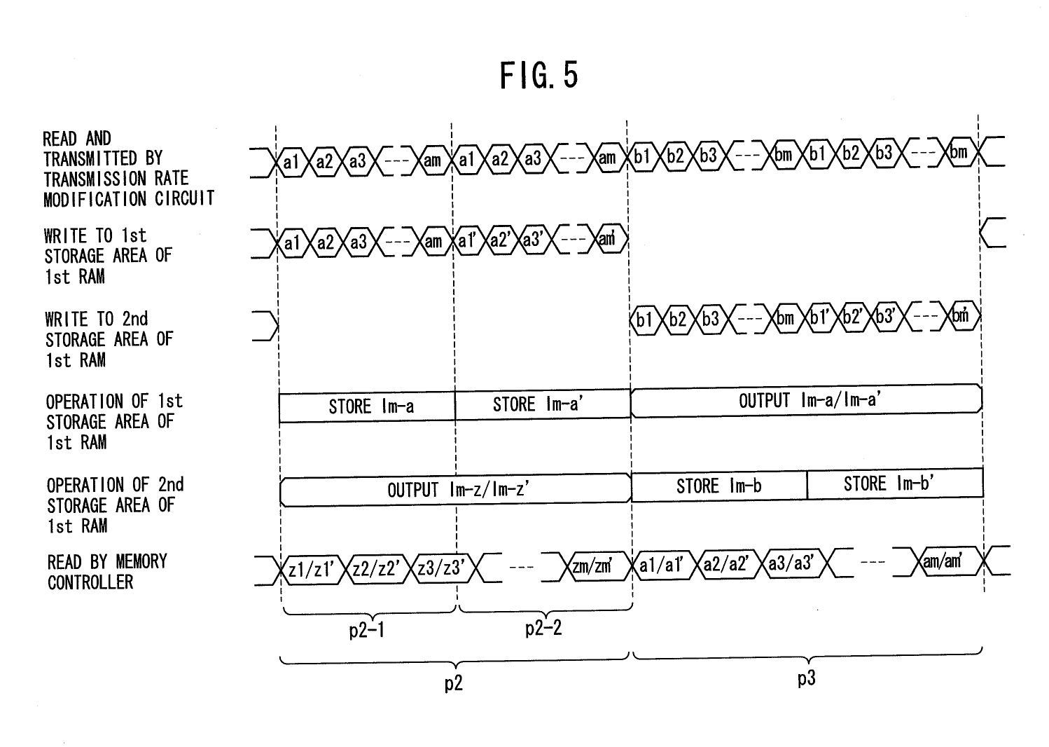

For example, the pixel signals that are transmitted from the transmission-rate modification circuit 28 during the period p2-1 (a1 through am in the row “Write to first storage area” in FIG. 5) are stored in the first storage area of the first RAM 48a in order. Furthermore, the pixel signals that are transmitted from the transmission-rate modification circuit 28 during the period p2-2 (a1′ through am′ in the row “Write to first storage area” in FIG. 5) are also stored in the first storage area of the first RAM 48a, in order. Accordingly, Im-a, which is the ath frame of the image signal transmitted first, and Im-a′, which is the ath frame of the image signal transmitted second, are stored in the first storage area of the first RAM 48a (see the row “Operation of first storage area” in FIG. 5).

During period p3, Im-b and Im-b′, which are the bth frame of the image signals transmitted from the transmission-rate modification circuit 28 first and second, respectively, are stored in the second storage area of the first RAM 48a (see the row “Operation of second storage area” in FIG. 5).

After the same frame of an image signal is stored in one of the storage areas of the first RAM 48a twice, a combination of both copies of each pixel, stored separately in the first or second storage areas, is read by the memory controller 47, and output to the determination circuit 49.

For example, the combinations of pixel signal pairs, corresponding to the first through mth pixels, which Im-a and Im-a′ comprises, written to the first storage area during period p2, are output to the determination circuit 29 in order via the memory controller 47 (see a1/a1′ through am/am′ in the row “Read by memory controller” in FIG. 5).

The determination circuit 49 compares the signal levels of pairs of received pixel signals. If the signal levels are equal, the determination circuit 29 determines that noise is not mixed into the pixel signal. On the other hand, if the signal levels are different, the determination circuit 49 determines that noise is mixed into the pixel signal.

The determination circuit 49 transmits a parity bit indicating the determination, together with the pixel signal, to the interpolation circuit 50. It signals that noise has not been mixed into the pixel signal with a parity bit of zero. It signals that noise has been mixed into the pixel signal with a parity bit of one. Incidentally, the pixel signal transmitted from the determination circuit 49 to the interpolation circuit 50 may be either of the first or second pixel signal copies transmitted from the electronic endoscope.

The interpolation circuit 50 writes the received pixel signal to the second RAM 48b based on the received parity bit. If the parity bit is one, the received pixel signal is discarded. Accordingly, if the signal levels of pairs of received pixel signals are different, the received pixel signal is discarded. If the parity bit is zero, the pixel signal is written to the second RAM 48b. Accordingly, if the signal levels of both copies in a pair of received pixel signals are equal, the received pixel signal is written to the second RAM 48b.

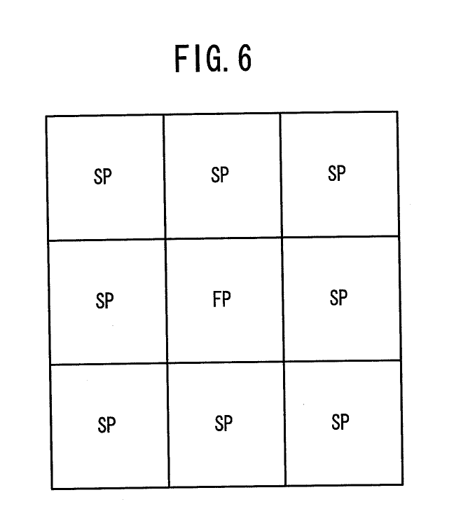

Furthermore, the interpolation circuit carries out interpolation processing for the “focused” pixel, that is, the pixel whose pixel signal was discarded. The eight pixels surrounding the focused pixel (FP in FIG. 6) are used for interpolation processing. In interpolation processing, a pixel signal corresponding to the focused pixel is generated using the eight pixel signals of the surrounding pixels (SP in FIG. 6). For example, the pixel signal of the focused pixel is generated in a smoothing operation by averaging the pixel signals of the eight surrounding pixels. Alternatively, the pixel signal of the focused pixel may be generated through the median filter processing by calculating the median of the surrounding eight pixel signals. The pixel signal of the focused pixel generated by interpolation processing is written to the second RAM 48b.

After finishing interpolation processing for all pixels whose pixel signals in one frame of an image signal were discarded, all pixel signals stored in the second RAM 48b are output as an image signal to the interpolation circuit 50, and further output to the second signal-processing circuit 46b.

The second signal-processing circuit 46b carries out predetermined signal processing, such as clamp processing and blanking processing on the received image signal. In addition, the second signal-processing circuit 46b converts the image signal from digital to analog. The image signal converted into an analog signal is sent to the monitor 11, where an image corresponding to the received image signal is displayed.

The system controller 45 controls each of components of the endoscope processor 40. The timing generator 44 synchronizes the timing of each component.

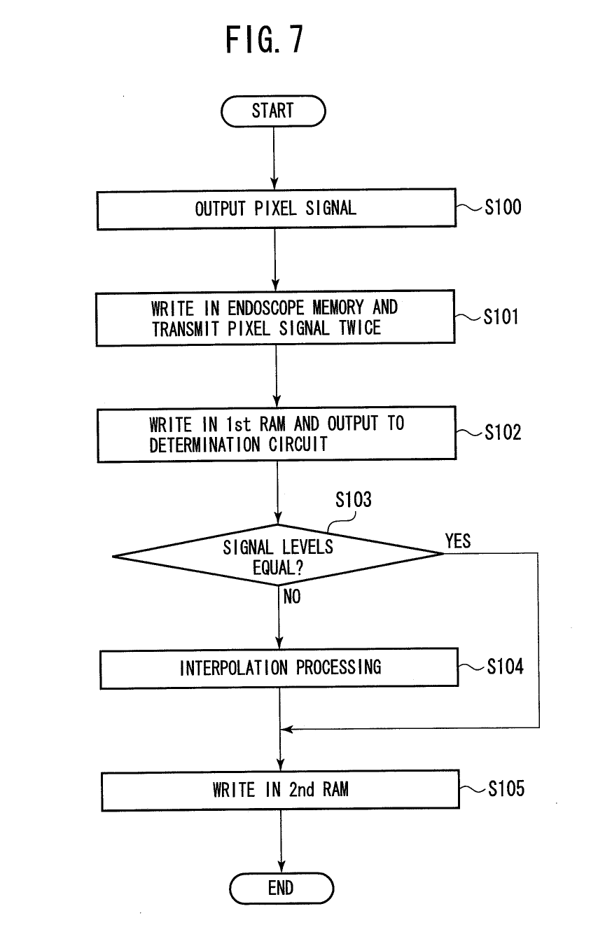

Next, the noise-reduction processing performed by the system controller 45 and the timing generator 44 to reduce the influence of noise in the first embodiment is explained below, using the flowchart of FIG. 7.

At step S100, the imaging device driver 27 is ordered to drive the imaging device 26 so that the imaging device 26 outputs pixel signals at a 13.5 MHz transmission rate. At step S101, the transmission-rate modification circuit 28 is ordered to write the pixel signals output from the imaging device 26 to the endoscope memory 29. In addition, the transmission-rate modification circuit 28 is ordered to transmit the stored pixel signals to the endoscope processor 40 at a 27 MHz transmission rate twice, at different times.

At step S102, the memory controller 47 is ordered to write the image signal transmitted from the electronic endoscope 20 twice, at different times, to the first RAM 48a. At the same time, the memory controller 47 is ordered to output the image signal stored in the first RAM 48a to the determination circuit 49 by outputting combinations of same-pixel signal pairs for the first pixel to the mth pixel, in order.

At step S103, the determination circuit 49 is ordered to compare the signal levels of the pixel pairs. When the two signal levels differ, the process proceeds to step S104. When the two signal levels are equal, the process skips step S104 and proceeds to step S105.

At step S104, the interpolation circuit 50 is ordered to discard the pixel signal and to carry out interpolation processing. After interpolation processing, the process proceeds to step S105.

At step S105, the interpolation circuit 50 is ordered to write the pixel signal which is generated according to interpolation processing at step S104 or is used for comparison at step S103 in the second RAM 48b. After writing the pixel signal to the second RAM 48b, the noise-reduction process ends.

In the first embodiment above, a pixel assumed to include noise is detected and a new pixel signal is generated for the detected pixel through interpolation. Thus, it is possible to reduce the noise mixed into a transmitted pixel signal.

Next, the image-signal transmission system of the second embodiment is explained. The primary difference between the second embodiment and the first embodiment, which is explained below, is the transmission rate at which the transmission-rate modification circuit transmits an image signal, and the type of signal processing performed on a pixel signal determined to be noisy. Here, the same index numbers are used for the structures corresponding to those of the first embodiment.

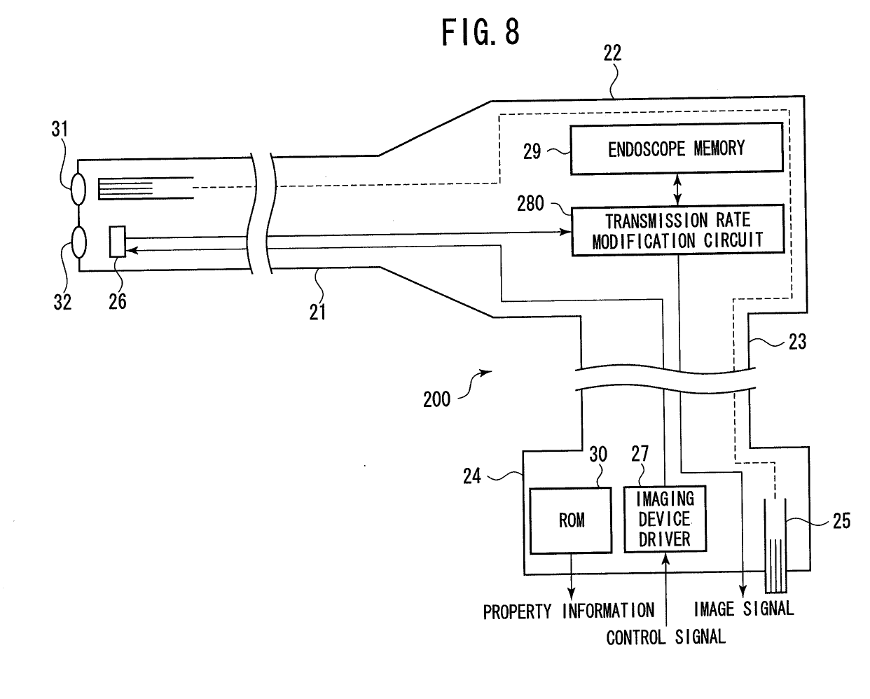

As shown in FIG. 8, the electronic endoscope 200 comprises a light guide 25, an imaging device 26, an imaging device driver 27, a transmission-rate modification circuit 280, an endoscope memory 29, a ROM 30, and other components as in the first embodiment. The structures and functions of the light guide 25, the imaging device 26, the imaging device driver 27, the endoscope memory 29 and the ROM 30 of the second embodiment are the same as those of the first embodiment.

The transmission rate at which the transmission-rate modification circuit 280 outputs pixel signals and the multiple used to transmit the pixel signal copies differ from those of the first embodiment. The transmission-rate modification circuit 280 outputs pixel signals at a 40.5 MHz transmission rate, which is three times the transmission rate at which the imaging device 26 outputs pixel signals, to the endoscope processor 40. In addition, the transmission-rate modification circuit 280 outputs the same pixel signal three times.

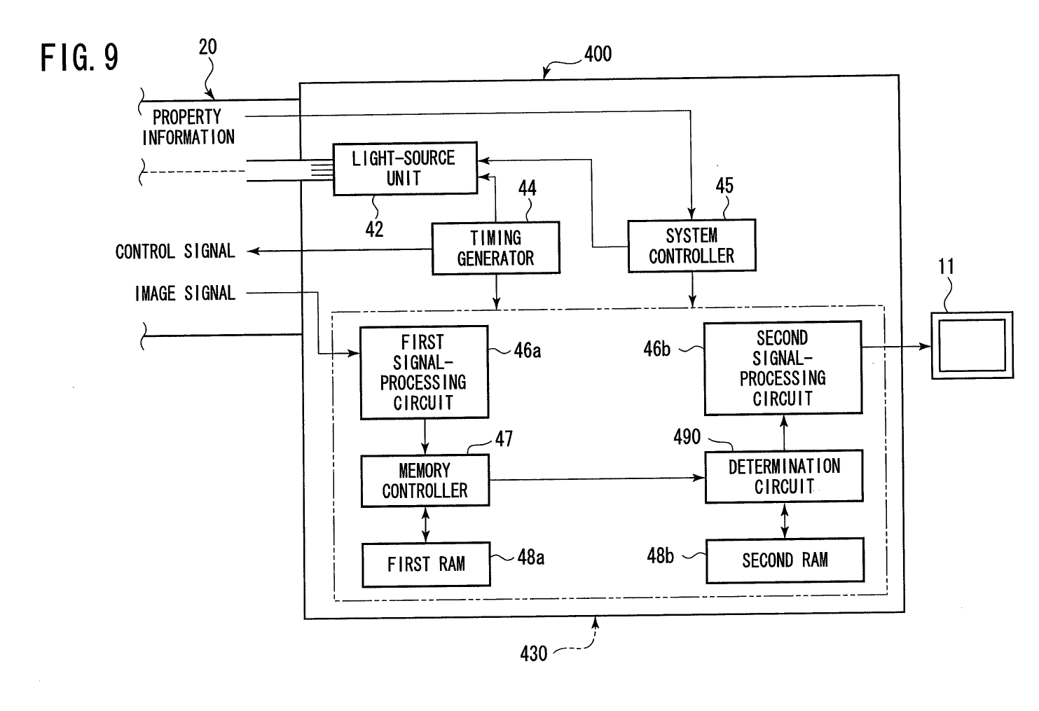

As shown in FIG. 9, the endoscope processor 400 comprises a light-source unit 42, a signal-processing unit 430, a timing generator 44, a system controller 45, and other components, as in the first embodiment. The structures and functions of the light-source unit 42, the timing generator 44, and the system controller 45 of the second embodiment are the same as those of the first embodiment.

The structure of the signal-processing unit 430 is different from that of the first embodiment. The signal-processing unit 430 comprises first and second signal-processing circuits 46a and 46b, a memory controller 47, first and second RAMs 48a and 48b, and a determination circuit 490.

The pixel signals transmitted from the electronic endoscope 200 to the signal-processing unit 430 is received by the first signal-processing circuit 46a, which carries out predetermined signal processing on the received pixel signals as in the first embodiment. The pixel signals, having undergone predetermined signal processing, are output to the memory controller 47.

The memory controller 47 writes the received pixel signals to the first RAM 48a as in the first embodiment. As described above, the pixel signals for a given frame of an image signal are transmitted three times from the electronic endoscope 200. The first RAM 48a has first and second storage areas which can store three frames of an image signal. The pixel signals transmitted three times are stored in either the first or the second storage areas. A combination of three pixel signals, originating from the same pixel, in three separately stored image signals in the first or second storage area is read by the memory controller 47, and output to the determination circuit 490 as in the first embodiment.

The determination circuit 490 compares the signal levels of the three received pixel signals as in the first embodiment. If all of the signal levels are equal, the determination circuit 290 determines that noise is not mixed into the pixel signal. On the other hand, if the signal levels are not all equal, the determination circuit 490 determines that noise is mixed into the pixel signal.

The second RAM 48b communicates with the determination circuit 490, unlike in the first embodiment. If the determination circuit 490 determines that noise is not mixed into the pixel signal, the determination circuit 490 writes the pixel signal to the second RAM 48b.

If the determination circuit 490 determines that noise is mixed into the pixel signal, the determination circuit 490 then determines whether the signal levels of two among the three pixel signals are equal. If the signal levels of two pixel signals are equal, the determination circuit 490 selects the two pixel signals with equal signal levels and writes one of them to the second RAM 48b. If the levels of all three pixel signals are different, the determination circuit 490 averages the three pixel signals and writes the averaged pixel signal to the second RAM 48b.

After writing all pixel signals for one frame of an image signal to the second RAM 48b, the determination circuit 490 reads all the pixel signals stored in the second RAM 48b as an image signal and outputs the image signal to the second signal-processing circuit 46b.

The second signal-processing circuit 46b carries out predetermined signal processing on the received image signal as in the first embodiment. In addition, the second signal-processing circuit 46b converts the image signal from digital to analog. The converted image signal is sent to the monitor 11, where an image corresponding to the received image signal is displayed.

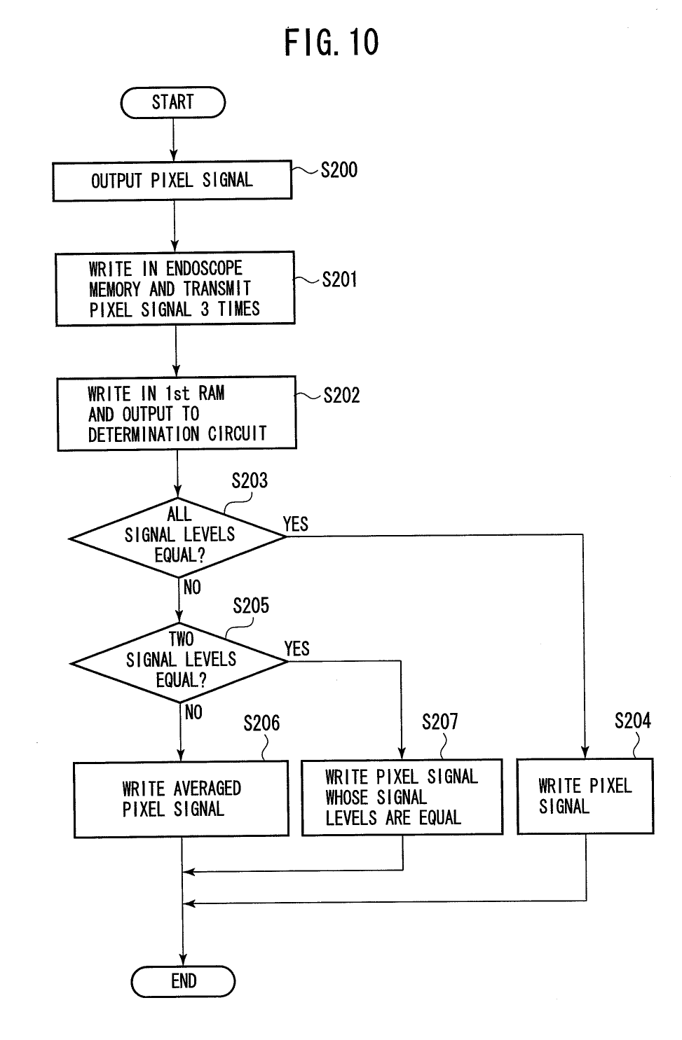

Next, the noise-reduction processing performed by the system controller 45 and the timing generator 44 in the second embodiment is explained, using the flowchart of FIG. 10.

At step S200, the imaging device driver 27 is ordered to drive the imaging device 26 so that the imaging device 26 outputs pixel signals at a 13.5 MHz transmission rate, as in the first embodiment. At step S201, the transmission-rate modification circuit 280 is ordered to write the pixel signal output from the imaging device 26 to the endoscope memory 28. In addition, the transmission-rate modification circuit 280 is ordered to transmit the stored pixel signals to the endoscope processor 40 at a 40.5 MHz transmission rate three times, successively.

At step S202, the memory controller 47 is ordered to write the image signal transmitted from the electronic endoscope 20 three times, successively, to the first RAM 48a. At the same time, the memory controller 47 is ordered to output the image signal stored in the first RAM 48a to the determination circuit 490 by outputting combinations of three pixel signal copies from the first to the mth pixel, in order.

At step S203, the determination circuit 490 is ordered to determine whether the signal levels of all three pixel signals of the same pixel are equal. When the signal levels of all three pixel signals are equal, the process proceeds to step S204. When the signal levels of all three pixel signals are not equal, the process proceeds to step S205.

At step S204, the determination circuit 490 is ordered to write the pixel signal used for comparison at step S203 to the second RAM 48b.

At step S205, the determination circuit 490 is ordered to determine whether the signal levels of two among the three pixel signals of the same pixel are equal. When the signal levels of all three pixel signals are different, the process proceeds to step S206. When the signal levels of two pixel signals are equal, the process proceeds to step S207.

At step S206, the determination circuit 490 is ordered to average the three pixel signals for the same pixel and write the averaged pixel signal to the second RAM 48b.

At step S207, the determination circuit 490 is ordered to write the pixel signal whose signal levels are equal to the second RAM 48b.

After writing the pixel signal to the second RAM 48b at step S204, S206, or S207, the noise-reduction process ends.

In the second embodiment above, either a pixel assumed to be noisy is detected, and a new pixel signal with reduced noise is generated for the detected pixel, or a noise free pixel signal is selected as the detected pixel. Accordingly, it is possible to reduce the noise mixed into a transmitted pixel signal.

In addition, as for the second embodiment, even if noise is mixed into one of the three pixel signals, it is possible to select another pixel signal which is noise free, unlike in the first embodiment. Because the selected pixel signal is used, noise is reduced further than in the first embodiment. However, the transmission-rate modification circuit 280 needs to multiply the transmission rate by a factor of three or more as compared to the imaging device 26, and needs to output the same pixel signals three or more times, unlike in the first embodiment. Consequently, a more expensive transmission-rate modification circuit is necessary.

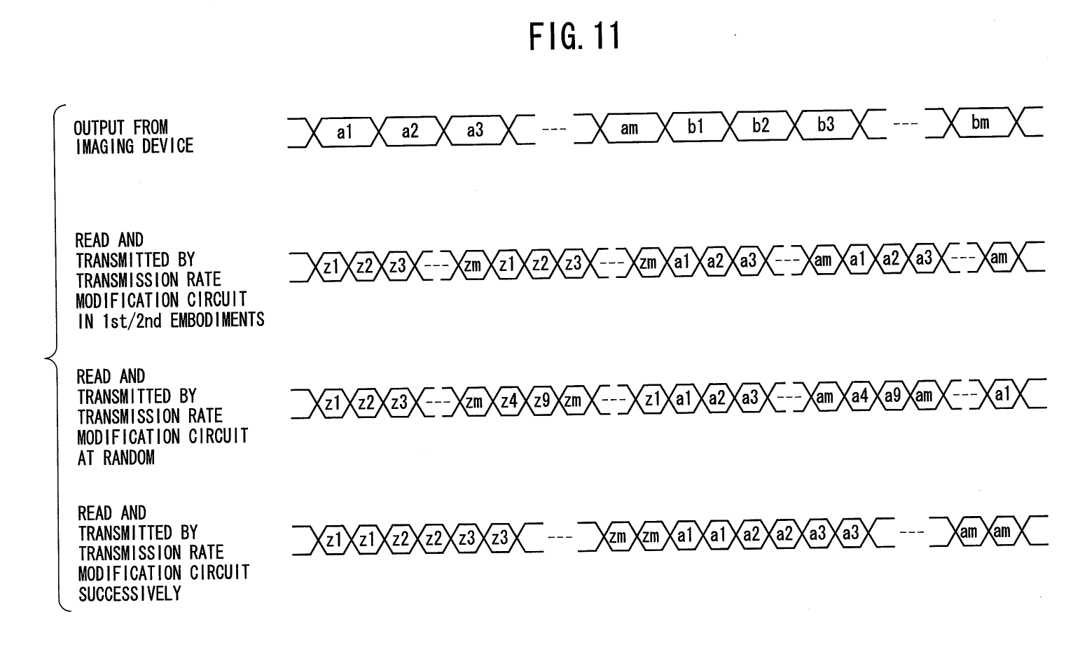

In the first and second embodiments, the transmission-rate modification circuits 28 and 280 transmit the pixel signals stored in the endoscope memory 29 in the same order as the imaging device 26 outputs them. However, the pixel signals can be transmitted from the transmission-rate modification circuit in any order as long as pixel signals of the same pixel are output multiple times during one frame period (see the row “Read and transmitted by transmission-rate modification circuit at random” in FIG. 11). As long as the transmission order of the pixel signals from the transmission-rate modification circuit is stored in the ROM 30 and the endoscope processor 40 recognizes that order, the signal-processing unit 40 can carry out noise reduction properly.

Alternatively, the pixel signal copies may be transmitted successively (see the row “Read and transmitted by transmission-rate modification circuit successively” in FIG. 11). By transmitting the same pixel signals successively, the endoscope memory is unnecessary. However, if the period in which noise may contaminate the pixel signals is longer than the period that the transmission-rate modification circuit 28 or 280 takes to transmit one pixel signal, then some pixel signals for the same pixel transmitted several times may include the same noise. In this case, noise would not be sufficiently reduced. Accordingly, it is preferable not to output the same pixel signal successively, especially when it is possible to implement the endoscope memory.

In the first and second embodiments, the transmission-rate modification circuits 28 and 280 are mounted in the control block 22. However, the transmission-rate modification circuit may be mounted at the head end of the insertion tube 21. If the transmission-rate modification circuits 28 and 280 are mounted in the head end of the insertion tube 21, the influence of noise generated between the insertion tube 21 and the control block 22 can be reduced. Consequently, if the transmission-rate modification circuits 28 and 280 can be miniaturized enough, it would be preferable to mount the transmission-rate modification circuits 28 or 280 at the head end of the insertion tube 21. Alternatively, the transmission-rate modification circuits 28 and 280 may be mounted in the connector 24. Even if it is mounted in the connector 24, the influence of noise generated between the connector 24 and the endoscope processor 40 can be reduced.

In the first and second embodiments, the memory controller 47, the first and second RAMs 48a and 48b, the determination circuits 49 and 490, and the interpolation circuit 50 are mounted in the endoscope processor 40. However, they may be mounted anywhere as long as they are mounted nearer the endoscope processor 40 than the transmission-rate modification circuits 28 or 280. By locating the point in the transmission path where noise is likely to be mixed into the signal passing from the transmission-rate modification circuit to the memory controller 47, and subsequent circuits, the influence of noise can be decreased.

In the first embodiment, interpolation processing is carried out using the pixel signals of the eight pixels surrounding the focused pixel. However, interpolation processing can be carried out by using pixel signals of subset of pixels surrounding the focused pixel. For example, pixel signals of the pixels above and below the focused pixel, or pixel signals of the pixels on the right or left side of the focused pixel, or pixel signals of the pixels at the upper, lower, right and left sides of the focused pixel, or pixel signals of the four pixels located diagonally from the focused pixel can be used for interpolation processing.

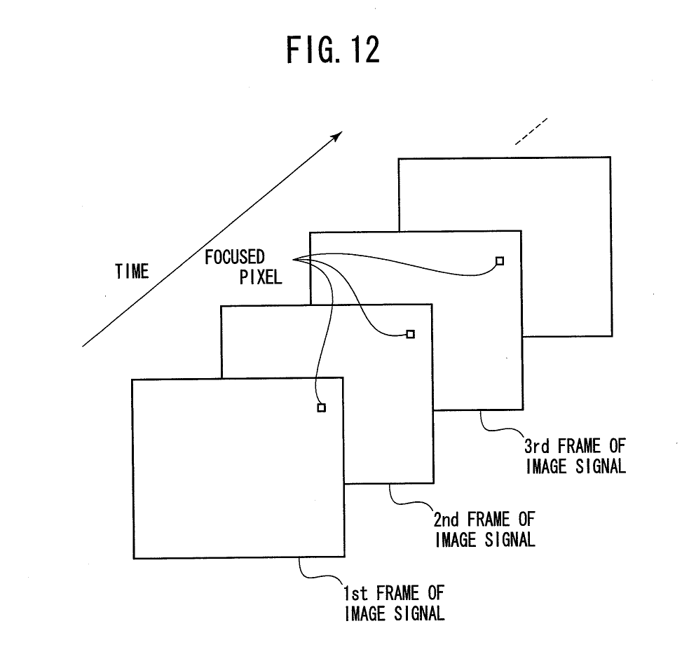

In addition, interpolation processing may be carried out using pixel signals not from the pixels surrounding the focused pixel but from the focused pixel outputs in the frame periods before and after the frame period in which noise was detected in the focused period. For example, as shown in FIG. 12, interpolation processing for the focused pixel generated during the second frame period can be carried out using pixel signals of the focused pixel generated during the first and third frame period.

In the first embodiment, the transmission-rate modification circuit 28 outputs pixel signals at a transmission rate twice as high as that of the imaging device 26. However, the transmission rate of the transmission-rate modification circuit 28 may be tow or more times higher than that of the imaging device 26. Likewise, the number of transmissions is not limited to two. The same pixel signal can be output two or more times from the transmission-rate modification circuit 28.

In the second embodiment, the transmission-rate modification circuit 280 outputs pixel signals at a transmission rate three times as high as the imaging device 26 does. However, the transmission rate of the transmission-rate modification circuit 280 can be three or more times as fast as that of the imaging device 26. Likewise, the number of transmissions is not limited to three. The same pixel signal can be output three or more times from the transmission-rate modification circuit 280.

Although the embodiments of the present invention have been described herein with reference to the accompanying drawings, obviously many modifications and changes may be made by those skilled in this art without departing from the scope of the invention.

The present disclosure relates to subject matter contained in Japanese Patent Application No. 2006-338281 (filed on Dec. 15, 2006), which is expressly incorporated herein, by reference, in its entirety.

1. An image-signal transmission system comprising:
an imaging device that outputs first pixel signals at a first frequency, said first pixel signals varying according to the amount of light received by pixels, a plurality of said pixels being arranged in two dimensions on the light-receiving surface of said imaging device;
a transmitter that transmits the first pixel signal copies separately several times at a second frequency, said second frequency being two or more times higher than said first frequency; and
a determination block that receives said first pixel signal copies from said transmitter several times and determines whether noise is mixed into said received first pixel signals by comparing said first pixel signal copies.

2. An image-signal transmission system according to claim 1, wherein said determination block determines that said noise is mixed into said received first pixel signal if the signal levels of a plurality of said first pixel signal copies are different.

3. An image-signal transmission system according to claim 1, further comprising an interpolation block that generates a second pixel signal using said received first pixel signals corresponding to said pixels surrounding a focused pixel, said focused pixel being a pixel which said determination block determines is noisy, said interpolation block replacing said received first pixel signal by said second pixel signal.

4. An image-signal transmission system according to claim 1, further comprising an interpolation block that generates a third pixel signal using a fourth pixel signal from the same focused pixel, said focused pixel being a pixel whose first pixel signal was determined to be noisy by said determination block, said fourth pixel signal being generated before and/or after said first pixel signal, said interpolation block replacing said first pixel signal by said third pixel signal.

5. An image-signal transmission system according to claim 1, wherein said determination block outputs a fifth pixel signal among said first pixel signal copies of the same pixel where said determination block determines said noise is mixed into said received first pixel signal, said fifth pixel signal being one among a plurality of said first pixel signal copies, the number of said fifth pixel signals whose signal levels are substantially equal to each other being the greatest among said first pixel signal copies.

6. An image-signal transmission system according to claim 1, wherein said transmitter transmits said first pixel signal copies several times successively.

7. An image-signal transmission system according to claim 1, wherein said transmitter transmits said first pixel signal copies several times at predetermined intervals.

8. An image-signal transmission system according to claim 1, wherein the interval where said transmitter transmits said first pixel signal copies several times is different for said first pixel signal.

9. An image-signal transmission system according to claim 1, wherein said transmitter is mounted at the head end of the insertion tube of an electronic endoscope.

10. An image-signal transmission system according to claim 1, wherein said transmitter is mounted in a control block of an electronic endoscope.

11. An image-signal transmission system according to claim 1, wherein said determination block is mounted in a connector of an electronic endoscope, said connector being inserted into the endoscope processor.

12. An image-signal transmission system according to claim 1, wherein said determination block is mounted in an endoscope processor.

13. An electronic endoscope comprising:
an imaging device that outputs first pixel signals at a first frequency, said first pixel signals varying according to the amount of light received by pixels, a plurality of said pixels being arranged in two dimensions on the light-receiving surface of said imaging device; and
a transmitter that transmits the first pixel signal copies separately several times at a second frequency, said second frequency being two or more times higher than said first frequency.

14. An endoscope processor comprising:
a receiver that receives first pixel signal copies from the electronic endoscope, said electronic endoscope comprising an imaging device and a transmitter, said imaging device outputting said first pixel signal at a first frequency, said first pixel signal varying according to the amount of light received by pixels, a plurality of said pixels being arranged in two dimensions on the light-receiving surface of said imaging device, said transmitter transmitting said first pixel signal copies separately several times at a second frequency, said second frequency being two or more times higher than said first frequency; and
a determination block that determines whether noise is mixed into said first pixel signal by comparing said first pixel signal copies which are transmitted separately several times from said transmitter.
